(19) United States
(12) Patent Application Publication
(10) Pub. No.: US 2010/0311751 A1
(43) Pub. Date: Dec. 9, 2010

(54) SOLID DISPERSIONS CONTAINING AN APOPTOSIS-PROMOTING AGENT

(75) Inventors: Eric A. Schmitt, Libertyville, IL (US); Ping Tong, Libertyville, IL (US); Katherine Heemstra, Chicago, IL (US); Cristina M. Fischer, Wadsworth, IL (US); Huailiang Wu, Long Grove, IL (US); Jonathan Mark Miller, Lindenhurst, IL (US); Yanxia Li, Grayslake, IL (US); Justin S. Lafountaine, Chicago, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/796,061

(22) Filed: Jun. 8, 2010

Related U.S. Application Data
(60) Provisional application No. 61/185,105, filed on Jun. 8, 2009.

(51) Int. Cl. A61K 31/5377; A61P 35/00

(52) U.S. Cl. 514/235.8

(57) ABSTRACT

A pro-apoptotic solid dispersion comprises, in essentially non-crystalline form, a Bcl-2 family protein inhibitory compound, e.g., ABT-263, dispersed in a solid matrix that comprises (a) a pharmaceutically acceptable water-soluble polymeric carrier and (b) a pharmaceutically acceptable surfactant. A process for preparing such a solid dispersion comprises dissolving the compound, the polymeric carrier and the surfactant in a suitable solvent, and removing the solvent to provide a solid matrix comprising the polymeric carrier and the surfactant and having the compound dispersed in essentially non-crystalline form therein. The solid dispersion is suitable for oral administration to a subject in need thereof for treatment of a disease characterized by overexpression of one or more anti-apoptotic Bcl-2 family proteins, for example cancer.

This application claims priority benefit of U.S. provisional application Ser. No. 61/185,105 filed on Jun. 8, 2009.

### FIELD OF THE INVENTION

The present invention relates to solid dispersions comprising an apoptosis-promoting agent, to pharmaceutical dosage forms comprising such dispersions, to processes for preparing such dispersions and dosage forms and to methods of use thereof for treating diseases characterized by overexpression of anti-apoptotic Bcl-2 family proteins.

### BACKGROUND OF THE INVENTION

Evasion of apoptosis is a hallmark of cancer (Hanahan & Weinberg (2000) Cell 100:57-70). Cancer cells must overcome a continual bombardment by cellular stresses such as DNA damage, oncogene activation, aberrant cell cycle progression and harsh microenvironments that would cause normal cells to undergo apoptosis. One of the primary means by which cancer cells evade apoptosis is by up-regulation of anti-apoptotic proteins of the Bcl-2 family.

Compounds that occupy the BH3 binding groove of Bcl-2 proteins have been described, for example by Bruncko et al. (2007) J. Med. Chem. 50:641-662. These compounds have included N-(4-(4-((4′-chloro-(1,1′-biphenyl)-2-yl)methyl)piperazin-1-yl)benzoyl)-4-(1R)-3-(dimethylamino)-1-((phenylsulfanyl)methyl)propyl)amino)-3-nitrobenzene-sulfonamide, otherwise known as ABT-737, which has the formula:

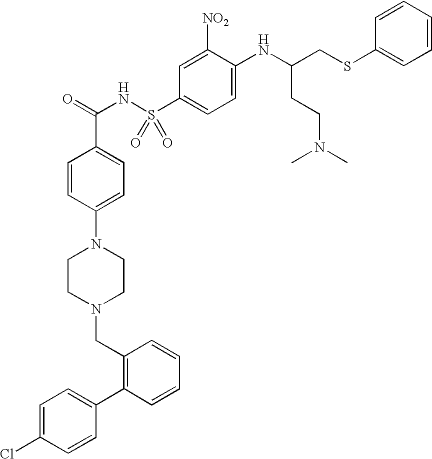

ABT-737 binds with high affinity (Kᵢ<1 nM) to proteins of the Bcl-2 family (specifically Bcl-2, Bcl-X_{L} and Bcl-w). It exhibits single-agent activity against small-cell lung cancer (SCLC) and lymphoid malignancies, and potentiates pro-apoptotic effects of other chemotherapeutic agents. ABT-737 and related compounds, and methods to make such compounds, are disclosed in U.S. Patent Application Publication No. 2007/0072860 of Bruncko et al.

More recently, a further series of compounds has been identified having high binding affinity to Bcl-2 family proteins. These compounds, and methods to make them, are disclosed in U.S. Patent Application Publication No. 2007/0027135 of Bruncko et al. (herein “the '135 publication”), incorporated by reference herein in its entirety, and can be seen from their formula below to be structurally related to ABT-737.

The '135 publication states that while inhibitors of Bcl-2 family proteins previously known may have either potent cellular efficacy or high systemic exposure after oral administration, they do not possess both properties. A typical measure of cellular efficacy of a compound is the concentration eliciting 50% cellular effect (EC₅₀). A typical measure of systemic exposure after oral administration of a compound is the area under the curve (AUC) resulting from graphing plasma concentration of the compound versus time from oral administration. Previously known compounds, it is stated in the '135 publication, have a low AUC/EC₅₀ ratio, meaning that they are not orally efficacious. Compounds of the above formula, by contrast, are stated to demonstrate enhanced properties with respect to cellular efficacy and systemic exposure after oral administration, resulting in a AUC/EC₅₀ ratio significantly higher than that of previously known compounds.

One compound, identified as “Example 1” in the '135 publication, is N-(4-(4-(2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide, otherwise known as ABT-263. This compound has a molecular weight of 974.6 g/mol and has the formula:

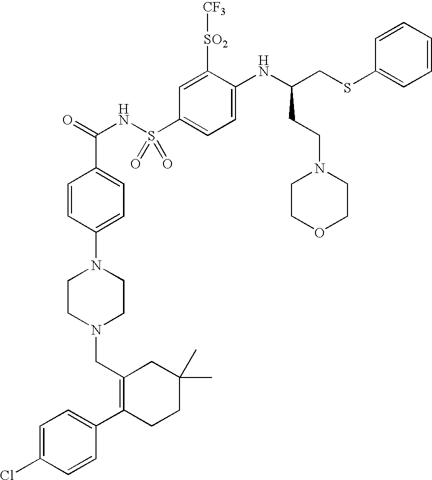

ABT-263 binds with high affinity (<1 nM) to Bcl-2 and Bcl-X_{L} and is believed to have similarly high affinity for Bcl-w. Its AUC/EC₅₀ ratio is reported in the '135 publication as 56, more than an order of magnitude greater than that reported for ABT-737 (4.5). For determination of AUC according to the '135 publication, each compound was administered to rats in a single 5 mg/kg dose by oral gavage as a 2 mg/ml solution in a vehicle of 10% DMSO (dimethyl sulfoxide) in PEG-400 (polyethylene glycol of average molecular weight about 400).

Oral bioavailability (as expressed, for example, by AUC after oral administration as a percentage of AUC after intravenous administration) is not reported in the '135 publication, but can be concluded therefrom to be substantially greater for ABT-263 than for ABT-737.

Recently, Tse et al. (2008) Cancer Res. 68(9):3421-3428, reported in supplementary data thereto that, in a dog model, oral bioavailability of an ABT-263 solution in PEG-400/DMSO was 22.4%, and that of an ABT-263 solution in 60% Phosal™ PG (phosphatidylcholine+propylene glycol), 30% PEG-400 and 10% ethanol was 47.6%.

Oxidation reactions represent an important degradation pathway of pharmaceuticals, especially when formulated in solution. Oxidation can occur by a number of pathways, including uncatalyzed autoxidation of a substrate by molecular oxygen, photolytic initiation, hemolytic thermal cleavage, and metal catalysis. Various functional groups show particular sensitivity towards oxidation. In particular, thioethers can degrade via hydrogen abstraction at the α-position to the sulfur atom or by addition of an α-peroxyl radical directly or via a one-electron transfer process, which transforms a sulfide to a sulfine, sulfone, or sulfoxide (Hovorka & Schoneich (2001) J. Pharm. Sci. 90:253-269).

The (phenylsulfanyl)methyl group possessed by compounds disclosed in the '135 publication, including ABT-263, is seen to have a thioether linkage, which is susceptible to oxidation, for example in presence of oxygen or reactive oxygen species such as superoxide, hydrogen peroxide or hydroxyl radicals. The '135 publication includes antioxidants in an extensive list of excipients said to be useful for administering the compounds disclosed therein.

However, pharmaceutical compositions that are less susceptible to oxidation of the active ingredient would be advantageous. Additionally, compositions capable of higher active ingredient loading than the solution compositions of the '135 publication or of Tse et al. (2008), supra would be advantageous. Further, liquid formulations as disclosed in the '135 publication and in Tse et al. (2008), supra can be unpleasant to take orally for taste or other reasons and can present patient compliance problems for these reasons; thus a solid-state composition would be beneficial.

The very low aqueous solubility of compounds of the '135 publication including ABT-263 raises challenges for the formulator, especially where there is a need to maintain acceptable oral bioavailability, which is strongly dependent on solubility in the aqueous medium of the gastrointestinal tract. Various solutions to the challenge of low oral bioavailability have been proposed in the art. For example, Sharma & Joshi (2007) Asian Journal of Pharmaceutics 1(1):9-19 discuss various solubility enhancement strategies in preparing solid dispersions. A solvent evaporation method for preparing solid dispersions is described therein, mentioning as an example a solid dispersion of etoricoxib, prepared by a process that includes dissolving polyethylene glycol (PEG), polyvinylpyrrolidone (PVP or povidone) and the active ingredient in 2-propanol.

A particular type of disease for which improved therapies are needed is non-Hodgkin's lymphoma (NHL). NHL is the sixth most prevalent type of new cancer in the U.S. and occurs primarily in patients 60-70 years of age. NHL is not a single disease but a family of related diseases, which are classified on the basis of several characteristics including clinical attributes and histology.

One method of classification places different histological subtypes into two major categories based on natural history of the disease, i.e., whether the disease is indolent or aggressive. In general, indolent subtypes grow slowly and are generally incurable, whereas aggressive subtypes grow rapidly and are potentially curable. Follicular lymphomas are the most common indolent subtype, and diffuse large-cell lymphomas constitute the most common aggressive subtype. The oncoprotein Bcl-2 was originally described in non-Hodgkin's B-cell lymphoma.

Treatment of follicular lymphoma typically consists of biologically-based or combination chemotherapy. Combination therapy with rituximab, cyclophosphamide, doxorubicin, vincristine and prednisone (R-CHOP) is routinely used, as is combination therapy with rituximab, cyclophosphamide, vincristine and prednisone (RCVP). Single-agent therapy with rituximab (targeting CD20, a phosphoprotein uniformly expressed on the surface of B-cells) or fludarabine is also used. Addition of rituximab to chemotherapy regimens can provide improved response rate and increased progression-free survival.

Radioimmunotherapy agents, high-dose chemotherapy and stem cell transplants can be used to treat refractory or relapsed NHL. Currently, there is not an approved treatment regimen that produces a cure, and current guidelines recommend that patients be treated in the context of a clinical trial, even in a first-line setting.

First-line treatment of patients with aggressive large B-cell lymphoma typically consists of rituximab, cyclophosphamide, doxorubicin, vincristine and prednisone (R-CHOP), or dose-adjusted etoposide, prednisone, vincristine, cyclophosphamide, doxorubicin and rituximab (DA-EPOCH-R).

Most lymphomas respond initially to any one of these therapies, but tumors typically recur and eventually become refractory. As the number of regimens patients receive increases, the more chemotherapy-resistant the disease becomes. Average response to first-line therapy is approximately 75%, 60% to second-line, 50% to third-line, and about 35-40% to fourth-line therapy. Response rates approaching 20% with a single agent in a multiple relapsed setting are considered positive and warrant further study.

Current chemotherapeutic agents elicit their antitumor response by inducing apoptosis through a variety of mechanisms. However, many tumors ultimately become resistant to these agents. Bcl-2 and Bcl-X_{L} have been shown to confer chemotherapy resistance in short-term survival assays in vitro and, more recently, in vivo. This suggests that if improved therapies aimed at suppressing the function of Bcl-2 and Bcl-X_{L} can be developed, such chemotherapy-resistance could be successfully overcome.

Apoptosis-promoting drugs that target Bcl-2 family proteins such as Bcl-2 and Bcl-X_{L} are best administered according to a regimen that provides continual, for example daily, replenishment of the plasma concentration, to maintain the concentration in a therapeutically effective range. This can be achieved by daily parenteral, e.g., intravenous (i.v.) or intraperitoneal (i.p.) administration. However, daily parenteral administration is often not practical in a clinical setting, particularly for outpatients. To enhance clinical utility of an apoptosis-promoting agent, for example as a chemotherapeutic in cancer patients, a solid dosage form with acceptable oral bioavailability would be highly desirable. Such a dosage form, and a regimen for oral administration thereof, would represent an important advance in treatment of many types of cancer, including NHL, and would more readily enable combination therapies with other chemotherapeutics.

### SUMMARY OF THE INVENTION

There is now provided a solid dispersion comprising, in essentially non-crystalline, for example amorphous, form, a compound of Formula I:

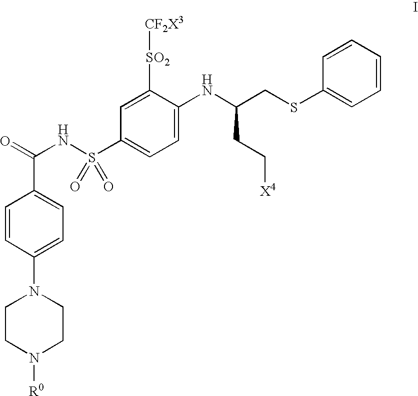

where:
X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is

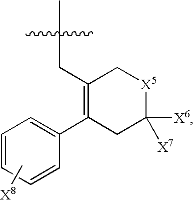

where
   X⁵ is —CH₂—, —C(CH₃)₂— or —CH₂CH₂;
   X⁶ and X⁷ are both —H or both methyl; and
   X⁸ is fluoro, chloro, bromo or iodo;
Or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is

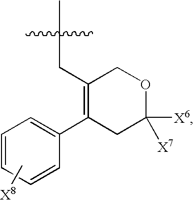

where X⁶, X⁷ and X⁸ are as above; or
(3) X⁴ is morpholin-4-yl or —N(CH₃)₂; and R⁰ is

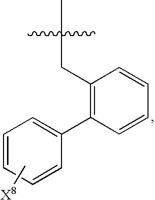

where X⁸ is as above;
   or a pharmaceutically acceptable salt, prodrug, salt of a prodrug or metabolite thereof; dispersed in a solid matrix that comprises (a) a pharmaceutically acceptable water-soluble polymeric carrier and (b) a pharmaceutically acceptable surfactant.

There is further provided a solid orally deliverable dosage form comprising such a solid dispersion, optionally together with one or more additional excipients.

There is still further provided a process for preparing a solid dispersion as described above. This process comprises:
(a) dissolving an active pharmaceutical ingredient (API) comprising (i) a compound of Formula I or a pharmaceutically acceptable salt, prodrug, salt of a prodrug or metabolite thereof, (ii) a pharmaceutically acceptable water-soluble polymeric carrier and (iii) a pharmaceutically acceptable surfactant in a suitable solvent; and
(b) removing the solvent to provide a solid matrix comprising the polymeric carrier and the surfactant and having the compound or a salt, prodrug, salt of a prodrug or metabolite thereof dispersed in an essentially non-crystalline form therein.

The compound present in the finished solid dispersion can be in the same chemical form (e.g., a free base or a salt) as in the API used to prepare it. Alternatively, the process comprises one or more additional steps wherein the compound is converted from free base to salt or vice versa. In a particular embodiment, the API is a salt, for example a crystalline salt, of a compound of Formula I, and the finished solid dispersion contains the compound in free base form. According to this embodiment, the process further comprises, prior to removing the solvent, adding a base for conversion of the salt to the free base, and optionally extracting a by-product of such conversion (e.g., a salt by-product) from the resulting mixture.

There is still further provided a solid dispersion prepared by the process described above.

There is still further provided a method for treating a disease characterized by apoptotic dysfunction and/or overexpression of an anti-apoptotic Bcl-2 family protein, comprising orally administering to a subject having the disease a therapeutically effective amount of a solid dispersion as described above, or one or more solid dosage forms comprising such a dispersion. Examples of such a disease include many neoplastic diseases including cancers. A specific illustrative type of cancer that can be treated according to the present method is non-Hodgkin's lymphoma (NHL). Another specific illustrative type of cancer that can be treated according to the present method is chronic lymphocytic leukemia. Yet another specific illustrative type of cancer that can be treated according to the present method is acute lymphocytic leukemia, for example in a pediatric patient.

According to any of the embodiments of the invention described above, the compound of Formula I is illustratively ABT-263 or a pharmaceutically acceptable salt, prodrug, salt of a prodrug or metabolite thereof, for example ABT-263 free base or ABT-263 bis-hydrochloride salt (ABT-263 bis-HCl).

There is still further provided a method for maintaining in bloodstream of a human cancer patient, for example a patient having NHL, a therapeutically effective plasma concentration of ABT-263 and/or one or more metabolites thereof, comprising orally administering to the subject a solid dispersion of ABT-263 or a pharmaceutically acceptable salt, prodrug, salt of a prodrug or metabolite thereof (for example ABT-263 free base or ABT-263 bis-HCl) in essentially non-crystalline form in a matrix that comprises a pharmaceutically acceptable water-soluble polymeric carrier and a pharmaceutically acceptable surfactant, in a dosage amount equivalent to about 50 to about 500 mg ABT-263 free base equivalent per day, at an average dosage interval of about 3 hours to about 7 days.

Additional embodiments of the invention, including more particular aspects of those provided above, will be found in, or will be evident from, the detailed description that follows.

### BRIEF DESCRIPTION OF THE DRAWINGS

FIG. 1 is a graphical representation of effects of various surfactants on dissolution rates of solid dispersions containing ABT-263 bis-HCl as described in Example 3.

FIG. 2 is a graphical representation of effects of various surfactants on dissolution rates of solid dispersions containing ABT-263 free base as described in Example 3.

FIG. 3 is a graphical representation of effects of various polymeric carriers on dissolution rates of solid dispersions containing ABT-263 bis-HCl as described in Example 4.

### DETAILED DESCRIPTION

A solid dispersion in accordance with the present disclosure comprises an active ingredient in an essentially non-crystalline or amorphous form, which is usually more soluble than the crystalline form. The term “solid dispersion” herein encompasses systems having small solid-state particles of one phase dispersed in another solid-state phase. More particularly, the present solid dispersions comprise one or more active ingredients dispersed in an inert carrier or matrix in solid state, and can be prepared by melting or solvent methods or by a combination of melting and solvent methods. According to the present invention a solvent method as described herein is particularly favored, avoiding the risk of thermal decomposition of the active ingredient by exposure to temperatures required to melt the polymeric carrier.

An “amorphous form” refers to a particle without definite structure, i.e., lacking crystalline structure.

The term “essentially non-crystalline” herein means that no more than about 5%, for example no more than about 2% or no more than about 1% crystallinity is observed by X-ray diffraction analysis. In a particular embodiment, no detectable crystallinity is observed by one or both of X-ray diffraction analysis or polarization microscopy.

Compounds of Formula I, including salts, prodrugs, salts of prodrugs and metabolites thereof, useful herein typically have very low solubility in water, for example less than about 100 μg/ml, in most cases less than about 30 μg/ml. The present invention can be especially advantageous for drugs that are essentially insoluble in water, i.e., having a solubility of less than about 10 μg/ml, since a process of the invention increases the apparent solubility of such a poorly-soluble active ingredient. Examples of such active ingredients are, for example, Biopharmaceutics Classification System (BCS) Class IV drug substances that are characterized by low solubility and low permeability (see “Waiver of in vivo bioavailability and bioequivalence studies for immediate-release solid oral dosage forms based on a biopharmaceutics classification system”, U.S. Department of Health and Human Services, Food and Drug Administration, Center for Drug Evaluation and Research (CDER), August 2000). It will be recognized that aqueous solubility of many compounds is pH-dependent; in the case of such compounds the solubility of interest herein is at a physiologically relevant pH, for example a pH of about 1 to about 8. Thus, in various embodiments, the drug has a solubility in water, at least at one point in a pH range from about 1 to about 8, of less than about 100 μg/ml, for example less than about 30 μg/ml, or less than about 10 μg/ml. Illustratively, ABT-263 has a solubility in water at pH 2 of less than 4 μg/ml.

Solid dispersions of the present invention comprise as active ingredient a compound of Formula I as defined above, or a pharmaceutically acceptable salt, prodrug, salt of a prodrug or metabolite of such a compound. Optionally they may further comprise a second active ingredient, for example a therapeutic agent useful in combination therapy with the compound of Formula I as indicated hereinbelow.

In one embodiment, the compound has Formula I where X³ is fluoro.

In a further embodiment, the compound has Formula I where X⁴ is morpholin-4-yl.

In a still further embodiment, the compound has Formula I where R⁰ is

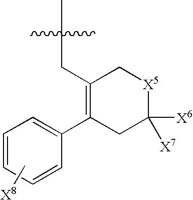

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

In a still further embodiment, the compound has Formula I where R⁰ is

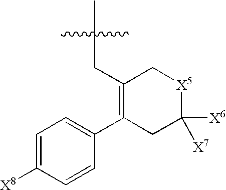

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

In a still further embodiment, the compound has Formula I where X³ is fluoro and X⁴ is morpholin-4-yl.

In a still further embodiment, the compound has Formula I where X³ is fluoro and R⁰ is

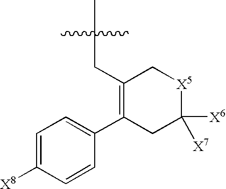

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

In a still further embodiment, the compound has Formula I where X⁴ is morpholin-4-yl and R⁰ is

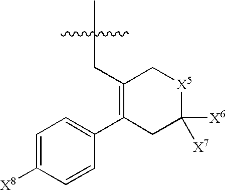

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

In a still further embodiment, the compound has Formula I where X³ is fluoro, X⁴ is morpholin-4-yl and R⁰ is

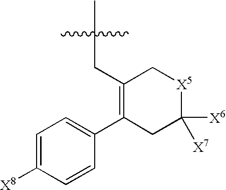

where X⁵ is —O—, —CH₂—, —C(CH₃)₂— or —CH₂CH₂—; X⁶ and X⁷ are both —H or both methyl; and X⁸ is fluoro, chloro, bromo or iodo. Illustratively according to this embodiment X⁵ can be —C(CH₃)₂— and/or each of X⁶ and X⁷ can be —H and/or X⁸ can be chloro.

Compounds of Formula I may contain asymmetrically substituted carbon atoms in the R- or S-configuration; such compounds can be present as racemates or in an excess of one configuration over the other, for example in an enantiomeric ratio of at least about 85:15. The compound can be substantially enantiomerically pure, for example having an enantiomeric ratio of at least about 95:5, or in some cases at least about 98:2 or at least about 99:1.

Compounds of Formula I may alternatively or additionally contain carbon-carbon double bonds or carbon-nitrogen double bonds in the Z- or E-configuration, the term “Z” denoting a configuration wherein the larger substituents are on the same side of such a double bond and the term “E” denoting a configuration wherein the larger substituents are on opposite sides of the double bond. The compound can alternatively be present as a mixture of Z- and E-isomers.

Compounds of Formula I may alternatively or additionally exist as tautomers or equilibrium mixtures thereof wherein a proton shifts from one atom to another. Examples of tautomers illustratively include keto-enol, phenol-keto, oxime-nitroso, nitro-aci, imine-enamine and the like.

In some embodiments, a compound of Formula I is present in the solid dispersion in its parent-compound form, alone or together with a salt or prodrug form of the compound.

Compounds of Formula I may form acid addition salts, basic addition salts or zwitterions. Salts of compounds of Formula I can be prepared during isolation or following purification of the compounds. Acid addition salts are those derived from reaction of a compound of Formula I with an acid. For example, salts including the acetate, adipate, alginate, bicarbonate, citrate, aspartate, benzoate, benzenesulfonate (besylate), bisulfate, butyrate, camphorate, camphorsulfonate, digluconate, formate, fumarate, glycerophosphate, glutamate, hemisulfate, heptanoate, hexanoate, hydrochloride, hydrobromide, hydroiodide, lactobionate, lactate, maleate, mesitylenesulfonate, methanesulfonate, naphthylenesulfonate, nicotinate, oxalate, pamoate, pectinate, persulfate, phosphate, picrate, propionate, succinate, tartrate, thiocyanate, trichloroacetate, trifluoroacetate, para-toluenesulfonate and undecanoate salts of a compound of Formula I can be used in a composition of the invention. Basic addition salts, including those derived from reaction of a compound with the bicarbonate, carbonate, hydroxide or phosphate of cations such as lithium, sodium, potassium, calcium and magnesium, can likewise be used.

A compound of Formula I typically has more than one protonatable nitrogen atom and is consequently capable of forming acid addition salts with more than one, for example about 1.2 to about 2, about 1.5 to about 2 or about 1.8 to about 2, equivalents of acid per equivalent of the compound.

ABT-263 can likewise form acid addition salts, basic addition salts or zwitterions. Salts of ABT-263 can be prepared during isolation or following purification of the compound. Acid addition salts derived from reaction of ABT-263 with an acid include those listed above. Basic addition salts including those listed above can likewise be used. ABT-263 has at least two protonatable nitrogen atoms and is consequently capable of forming acid addition salts with more than one, for example about 1.2 to about 2, about 1.5 to about 2 or about 1.8 to about 2, equivalents of acid per equivalent of the compound.

Illustratively in the case of ABT-263, bis-salts can be formed including, for example, bis-hydrochloride (bis-HCl) and bis-hydrobromide (bis-HBr) salts.

For example, ABT-263 bis-HCl, which has a molecular weight of 1047.5 g/mol and is represented by the formula

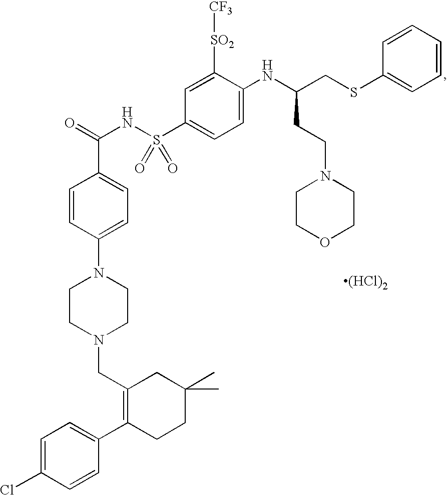

can be prepared by a variety of processes, for example a process that can be outlined as follows.

ABT-263 free base is prepared, illustratively as described in Example 1 of above-cited U.S. Patent Application Publication No. 2007/0027135, the entire disclosure of which is incorporated by reference herein. A suitable weight of ABT-263 free base is dissolved in ethyl acetate. A solution of hydrochloric acid in ethanol (for example about 4.3 kg HCl in 80 g EtOH) is added to the ABT-263 solution in an amount providing at least 2 mol HCl per mol ABT-263 and sufficient EtOH (at least about 20 vol) for crystallization of the resulting ABT-263 bis-HCl salt. The solution is heated to about 45° C. with stirring and seeds are added as a slurry in EtOH. After about 6 hours, the resulting slurry is cooled to about 20° C. over about 1 hour and is mixed at that temperature for about 36 hours. The slurry is filtered to recover a crystalline solid, which is an ethanol solvate of ABT-263 bis-HCl. Drying of this solid under vacuum and nitrogen with mild agitation for about 8 days yields white desolvated ABT-263 bis-HCl crystals. This material is suitable as an API for preparation of an ABT-263 bis-HCl or (by inclusion of a salt-to-base conversion step in the solid dispersion process) ABT-263 free base formulation of the present invention.

The term “free base” is used for convenience herein to refer to the parent compound, while recognizing that the parent compound is, strictly speaking, zwitterionic and thus does not always behave as a true base.

Compounds of Formula I, and methods of preparation of such compounds, are disclosed in above-cited U.S. Patent Application Publication No. 2007/0027135 and/or in above-cited U.S. Patent Application Publication No. 2007/0072860, each of which is incorporated herein by reference in its entirety. Terms for substituents used herein are defined exactly as in those publications.

Compounds of Formula I having —NH, —C(O)OH, —OH or —SH moieties may have attached thereto prodrug-forming moieties which can be removed by metabolic processes in vivo to release the parent compound having free —NH, —C(O)OH, —OH or —SH moieties. Salts of prodrugs can also be used.

Without being bound by theory, it is believed that the therapeutic efficacy of compounds of Formula I is due at least in part to their ability to bind to a Bcl-2 family protein such as Bcl-2, Bcl-X_{L} or Bcl-w in a way that inhibits the anti-apoptotic action of the protein, for example by occupying the BH3 binding groove of the protein. It will generally be found desirable to select a compound having high binding affinity for a Bcl-2 family protein, for example a Kᵢ not greater than about 5 nM, preferably not greater than about 1 nM.

A solid dispersion as provided herein comprising any specific compound disclosed in the '135 publication is expressly contemplated as an embodiment of the present invention.

In a more particular embodiment, the composition comprises ABT-263 or a salt, prodrug, salt of a prodrug or metabolite thereof. In a still more particular embodiment, the composition comprises ABT-263 parent compound (i.e., free base) or a salt, prodrug or salt of a prodrug thereof. In a still more particular embodiment, the composition comprises ABT-263 free base or a salt thereof. In an even more particular embodiment, the composition comprises ABT-263 free base or ABT-263 bis-HCl.

ABT-263 bis-HCl, by virtue of its crystalline nature, is typically more convenient to use as an API than ABT-263 free base, which as prepared according to the '135 publication is an amorphous or glassy solid. However, there may be advantages in providing a solid dispersion formulation of ABT-263 wherein the ABT-263 is in free base form, as the drug will be less susceptible to crystallization within the formulation or immediately upon release therefrom. Thus in a yet more particular embodiment, the composition comprises ABT-263 free base. It is emphasized that, in this embodiment, it is not necessarily the free base form of ABT-263 that is used as the API in preparing the composition.

A compound of Formula I or a salt, prodrug, salt of a prodrug or metabolite thereof) is present in a solid dispersion of the invention in an amount that can be therapeutically effective when the composition is administered to a subject in need thereof according to an appropriate regimen. Dosage amounts are expressed herein as parent-compound-equivalent (free base equivalent) amounts unless the context requires otherwise. Typically, a unit dose (the amount administered at a single time), which can be administered at an appropriate frequency, e.g., twice daily to once weekly, is about 10 to about 1,000 mg, depending on the compound in question. Where frequency of administration is once daily (q.d.), unit dose and daily dose are the same. Illustratively, for example where the drug is ABT-263, the unit dose is typically about 25 to about 1,000 mg, more typically about 50 to about 500 mg, for example about 50, about 100, about 150, about 200, about 250, about 300, about 350, about 400, about 450 or about 500 mg. Where the dosage form comprises a capsule shell enclosing the solid dispersion, or a tablet wherein the solid dispersion is formulated with other ingredients, a unit dose can be deliverable in a single dosage form or a plurality of dosage forms, most typically 1 to about 10 dosage forms.

The higher the unit dose, the more desirable it becomes to prepare a solid dispersion having a relatively high concentration of the drug therein. Typically, the concentration of drug in the solid dispersion is at least about 1%, e.g., about 1% to about 50%, by free base equivalent weight, but lower and higher concentrations can be acceptable or achievable in specific cases. Illustratively, for example where the drug is ABT-263, the drug concentration in various embodiments is at least about 2%, e.g., about 2% to about 50%, or at least about 5%, e.g., about 5% to about 40%, for example about 5%, about 10%, about 15%, about 20%, about 25%, about 30%, about 35% or about 40%, by free base equivalent weight.

The major component of the matrix of a solid dispersion product is a polymer that is hydrophilic or water-soluble at least in a part of the pH scale, more particularly at a pH occurring in the gastrointestinal (GI) tract, or a combination of such polymers. A polymer or polymer mixture useful herein is solid at ambient temperature and, in the interests of good storage stability at a range of temperatures, should remain solid even at the highest temperatures typically experienced during storage, transport and handling of the product. A useful property of a polymer determining its usefulness herein is therefore its glass transition temperature (T_{g}). Suitable water-soluble polymers include, but are not limited to, those having a T_{g} of at least about 50° C., more particularly about 80° C. to about 180° C. Methods for determining T_{g} values of organic polymers are described for example in Sperling, ed. (1992) Introduction To Physical Polymer Science, 2nd edition, John Wiley & Sons, Inc.

Non-limiting examples of polymeric carriers useful herein include:
homopolymers and copolymers of N-vinyl lactams, especially homopolymers and copolymers of N-vinyl pyrrolidone, e.g., the homopolymer polyvinylpyrrolidone (PVP or povidone) and copolymers such as those comprising monomers of N-vinyl pyrrolidone and vinyl acetate (copovidone) or N-vinyl pyrrolidone and vinyl propionate;
cellulose esters and cellulose ethers, in particular methylcellulose, ethylcellulose, (hydroxyalkyl)celluloses such as hydroxypropylcellulose, (hydroxyalkyl)alkyl-celluloses such as hydroxypropylmethylcellulose (HPMC or hypromellose), cellulose phthalates and succinates such as cellulose acetate phthalate, hydroxypropylmethylcellulose phthalate, hydroxypropylmethylcellulose succinate and hydroxypropylmethylcellulose acetate succinate (HPMC-AS);
high molecular weight polyalkylene oxides such as polyethylene oxide, polypropylene oxide and copolymers of ethylene oxide and propylene oxide (poloxamers);
polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates) and poly(hydroxyalkyl methacrylates);
polyacrylamides;
vinyl acetate polymers such as copolymers of vinyl acetate and crotonic acid, partially hydrolyzed polyvinyl acetate (also referred to as partially saponified “polyvinyl alcohol”) and polyvinyl alcohol;
oligo- and polysaccharides such as carrageenans, galactomannans and xanthan gum;
   and mixtures of two or more thereof.

In one embodiment, the solid dispersion matrix comprises one or more polymeric carriers selected from the group consisting of copovidone, povidone and HPMC-AS. A particular example of a useful copovidone is one consisting of about 60% N-vinyl pyrrolidone and about 40% vinyl acetate monomers. A particular example of a useful povidone is one having a K-value (a measure of viscosity of an aqueous solution of the povidone) of about 30.

One or more polymeric carriers typically constitute in total about 20% to about 90%, for example about 40% to about 85%, by weight of the solid dispersion.

Upon oral administration and exposure to GI fluid, it is believed without being bound by theory that, through interplay between the polymeric carrier and a surfactant component of the solid dispersion, a suitable release rate and inhibition of crystallization or recrystallization of the active ingredient are provided, thereby permitting bioabsorption.

Particularly useful as surfactants herein are pharmaceutically acceptable non-ionic surfactants, especially those having a hydrophilic-lipophilic balance (HLB) value of about 12 to about 18, for example about 13 to about 17, or about 14 to about 16. The HLB system (see Fiedler (2002) Encyclopedia of Excipients, 5th edition, Aulendorf: ECV-Editio-Cantor-Verlag) attributes numeric values to surfactants, with lipophilic substances receiving lower HLB values and hydrophilic substances receiving higher HLB values.

Non-limiting examples of non-ionic surfactants useful herein include:
polyoxyethylene castor oil derivatives such as PEG-35 castor oil (e.g., Cremophor EL™ of BASF Corp. or equivalent product), PEG-40 hydrogenated castor oil (e.g., Cremophor RH™ 40 or equivalent product) and PEG-60 hydrogenated castor oil (e.g., Cremophor RH™ 60 or equivalent product);
fatty acid monoesters of sorbitan, for example sorbitan monooleate (e.g., Span™ 80 or equivalent product), sorbitan monostearate (e.g., Span™ 60 or equivalent product), sorbitan monopalmitate (e.g., Span™ 40 or equivalent product) and sorbitan monolaurate (e.g., Span™ 20 or equivalent product);
fatty acid monoesters of polyoxyethylene sorbitan (polysorbates) such as PEG-20 sorbitan monooleate (polysorbate 80, e.g., Tween™ 80 or equivalent product) PEG-20 sorbitan monostearate (polysorbate 60, e.g., Tween™ 60 or equivalent product), PEG-20 sorbitan monopalmitate (polysorbate 40, e.g., Tween™ 40 or equivalent product), or PEG-20 sorbitan monolaurate (polysorbate 20, e.g., Tween™ 20 or equivalent product);
poloxamers such as poloxamer 124, poloxamer 188, poloxamer 237, poloxamer 388 or poloxamer 407;
α-tocopheryl polyethylene glycol succinate (TPGS or vitamin E polyethylene glycol succinate, see U.S. National Formulary);
   and mixtures of two or more thereof.

One or more surfactants typically constitute in total about 2% to about 25%, for example about 5% to about 20%, by weight of the solid dispersion.

A dosage form of the invention can consist of, or consist essentially of, a solid dispersion as described above. However, in some embodiments a dosage form contains additional excipients and requires additional processing of the solid dispersion. For example, the solid dispersion can be ground to a powder and filled into a capsule shell or molded or compressed to form a tablet, with additional excipients as may be conventionally used in such dosage forms.

Thus orally deliverable solid dosage forms of the invention include but are not limited to capsules, dragees, granules, pills, powders and tablets. Excipients commonly used to formulate such dosage forms include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, preservatives, propellants, releasing agents, sterilizing agents, sweeteners, solubilizers and mixtures thereof. Examples of specific excipients include agar, alginic acid, aluminum hydroxide, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, talc, tragacanth, water, safflower oil, sesame oil, sodium carboxymethyl cellulose, sodium lauryl sulfate, sodium phosphate salts, soybean oil, sucrose, tetrahydrofurfuryl alcohol, and mixtures thereof.

A solvent process for preparing a solid dispersion as described above comprises dissolving the API, the polymeric carrier and the surfactant in a suitable solvent; and removing the solvent to provide the solid dispersion. Optionally, where the API is in salt form and it is desired to provide a solid dispersion of the drug in free base form, a base is added before solvent removal to effect conversion of the API to its corresponding free base. For example, where the API is ABT-263 bis-HCl, addition of a base such as sodium hydroxide (NaOH), potassium hydroxide (KOH), sodium bicarbonate (NaHCO₃), potassium bicarbonate (KHCO₃) or ammonium bicarbonate (NH₄HCO₃) in an amount of at least 2 moles per mole of API can result in conversion of the API to ABT-263 free base. The inorganic salt by-product, illustratively NaCl, KCl or NH₄Cl, can remain in the product or is optionally extracted before solvent removal.

In the dissolving step, the various components can be added in any order. For example, each ingredient can be added to the solvent separately and then dissolved therein. Alternatively, the polymeric carrier and/or surfactant can be pre-mixed with the API, and the resulting mixture then added to the solvent. However, it will generally be found convenient, when the process includes in situ salt-to-free base conversion, to first add the API salt and the base to the solvent, then (optionally after extraction of a salt by-product) add the polymeric carrier and surfactant.

In principle any solvent can be used so long as it is effective to dissolve the active ingredient, polymer carrier and surfactant. Non-limiting examples of solvents that can be useful include methanol, ethanol, acetone and mixtures thereof. Optionally a cosolvent can be included.

Where it is desired to extract a salt by-product such as NaCl, KCl or NH₄Cl prior to solvent removal, a solvent can be selected wherein the salt by-product is insoluble, thereby permitting extraction of the salt by-product by filtration.

Solvent removal can be accomplished using heat, vacuum or a combination thereof. If heat is used, it is generally preferable to avoid exceeding the glass transition temperature (T_{g}) of the polymeric matrix. For most purposes heating at a temperature of about 50° C. to about 80° C., for example about 55° C. to about 75° C., will be found suitable. After solvent removal, the resulting product is cooled (if necessary) to ambient temperature.

Further process details can be found in the illustrative processes of Examples 1 and 2 below.

The terms “orally deliverable”, “oral administration” and “orally administered” herein refer to administration to a subject per os (p.o.), that is, administration wherein the composition is immediately swallowed, for example with the aid of a suitable volume of water or other potable liquid. “Oral administration” is distinguished herein from intraoral administration, e.g., sublingual or buccal administration or topical administration to intraoral tissues such as periodontal tissues, that does not involve immediate swallowing of the composition.

The active ingredient form (e.g., free base or salt), the polymeric carrier(s), surfactant(s) and other optional ingredients should be selected, and relative amounts of these components should be used, to provide a solid dispersion or dosage form having acceptable bioabsorption when administered orally. Such bioabsorption can be evidenced, for example, by the pharmacokinetic (PK) profile of the solid dispersion or dosage form, more particularly by the Cₘₐₓ or AUC, for example AUC₀₋₂₄ or AUC_{0-∞} at a particular dose or over a range of doses. Illustratively, bioavailability can be expressed as a percentage, for example using the parameter F, which computes AUC for oral delivery of a test composition as a percentage of AUC for intravenous (i.v.) delivery of the drug in a suitable solvent, taking into account any difference between oral and i.v. doses.

Bioavailability can be determined by PK studies in humans or in any suitable model species. For present purposes, a dog model, as illustratively described in Example 5 below, is generally suitable. In various illustrative embodiments, where the drug is ABT-263, compositions of the invention exhibit oral bioavailability of at least about 15%, at least about 20%, at least about 25% or at least about 30%, up to or exceeding about 50%, in a dog model, when administered as a single dose of about 2.5 to about 10 mg/kg to fasting or non-fasting animals.

Compositions embraced herein, including compositions described generally or with specificity herein, are useful for orally delivering a drug that is a compound of Formula I or a pharmaceutically acceptable salt, prodrug, salt of a prodrug or metabolite thereof to a subject. Accordingly, a method of the invention for delivering such a drug to a subject comprises orally administering a composition as described above.

The subject can be human or non-human (e.g., a farm, zoo, work or companion animal, or a laboratory animal used as a model) but in an important embodiment the subject is a human patient in need of the drug, for example to treat a disease characterized by apoptotic dysfunction and/or overexpression of an anti-apoptotic Bcl-2 family protein. A human subject can be male or female and of any age. The patient is typically an adult, but a method of the invention can be useful to treat a childhood cancer such as leukemia, for example acute lymphocytic leukemia, in a pediatric patient.

The composition is normally administered in an amount providing a therapeutically effective daily dose of the drug. The term “daily dose” herein means the amount of drug administered per day, regardless of the frequency of administration. For example, if the subject receives a unit dose of 150 mg twice daily, the daily dose is 300 mg. Use of the term “daily dose” will be understood not to imply that the specified dosage amount is necessarily administered once daily. However, in a particular embodiment the dosing frequency is once daily (q.d.), and the daily dose and unit dose are in this embodiment the same thing.

What constitutes a therapeutically effective dose depends on the particular compound, the subject (including species and body weight of the subject), the disease (e.g., the particular type of cancer) to be treated, the stage and/or severity of the disease, the individual subject's tolerance of the compound, whether the compound is administered in monotherapy or in combination with one or more other drugs, e.g., other chemotherapeutics for treatment of cancer, and other factors. Thus the daily dose can vary within wide margins, for example from about 10 to about 1,000 mg. Greater or lesser daily doses can be appropriate in specific situations. It will be understood that recitation herein of a “therapeutically effective” dose herein does not necessarily require that the drug be therapeutically effective if only a single such dose is administered; typically therapeutic efficacy depends on the composition being administered repeatedly according to a regimen involving appropriate frequency and duration of administration. It is strongly preferred that, while the daily dose selected is sufficient to provide benefit in terms of treating the cancer, it should not be sufficient to provoke an adverse side-effect to an unacceptable or intolerable degree. A suitable therapeutically effective dose can be selected by the physician of ordinary skill without undue experimentation based on the disclosure herein and on art cited herein, taking into account factors such as those mentioned above. The physician may, for example, start a cancer patient on a course of therapy with a relatively low daily dose and titrate the dose upwards over a period of days or weeks, to reduce risk of adverse side-effects.

Illustratively, suitable doses of ABT-263 are generally about 25 to about 1,000 mg/day, more typically about 50 to about 500 mg/day or about 200 to about 400 mg/day, for example about 50, about 100, about 150, about 200, about 250, about 300, about 350, about 400, about 450 or about 500 mg/day, administered at an average dosage interval of about 3 hours to about 7 days, for example about 8 hours to about 3 days, or about 12 hours to about 2 days. In most cases a once-daily (q.d.) administration regimen is suitable.

An “average dosage interval” herein is defined as a span of time, for example one day or one week, divided by the number of unit doses administered over that span of time. For example, where a drug is administered three times a day, around 8 am, around noon and around 6 pm, the average dosage interval is 8 hours (a 24-hour time span divided by 3). If the drug is formulated as a discrete dosage form such as a tablet or capsule, a plurality (e.g., 2 to about 10) of dosage forms administered at one time is considered a unit dose for the purpose of defining the average dosage interval.

Where the drug compound is ABT-263, for example in the form of ABT-263 free base or ABT-263 bis-HCl, a daily dosage amount and dosage interval can, in some embodiments, be selected to maintain a plasma concentration of ABT-263 in a range of about 0.5 to about 10 μg/ml. Thus, during a course of ABT-263 therapy according to such embodiments, the steady-state peak plasma concentration (Cₘₐₓ) should in general not exceed about 10 μg/ml, and the steady-state trough plasma concentration (Cₘᵢₙ) should in general not fall below about 0.5 μg/ml. It will further be found desirable to select, within the ranges provided above, a daily dosage amount and average dosage interval effective to provide a Cₘₐₓ/Cₘᵢₙ ratio not greater than about 5, for example not greater than about 3, at steady-state. It will be understood that longer dosage intervals will tend to result in greater Cₘₐₓ/Cₘᵢₙ ratios. Illustratively, at steady-state, an ABT-263 Cₘₐₓ of about 3 to about 8 μg/ml and Cₘᵢₙ of about 1 to about 5 μg/ml can be targeted by the present method. Steady-state values of Cₘₐₓ and Cₘᵢₙ can be established in a human PK study, for example conducted according to standard protocols including but not limited to those acceptable to a regulatory agency such as the U.S. Food and Drug Administration (FDA).

Where the composition is in the form of a capsule, one to a small plurality of capsules can be swallowed whole, typically with the aid of water or other imbibable liquid to help the swallowing process. Suitable capsule shell materials include, without limitation, gelatin (in the form of hard gelatin capsules or soft elastic gelatin capsules), starch, carrageenan and HPMC.

As compositions of the present invention are believed to exhibit only a minor food effect, administration according to the present embodiment can be with or without food, i.e., in a non-fasting or fasting condition. It is generally preferred to administer the present compositions to a non-fasting patient.

Compositions of the invention are suitable for use in monotherapy or in combination therapy, for example with other chemotherapeutics or with ionizing radiation. A particular advantage of the present invention is that it permits once-daily oral administration, a regimen which is convenient for the patient who is undergoing treatment with other orally administered drugs on a once-daily regimen. Oral administration is easily accomplished by the patient him/herself or by a caregiver in the patient's home; it is also a convenient route of administration for patients in a hospital or residential care setting.

Combination therapies illustratively include administration of a composition of the present invention, for example such a composition comprising ABT-263, concomitantly with one or more of bortezomib, carboplatin, cisplatin, cyclophosphamide, dacarbazine, dexamethasone, docetaxel, doxorubicin, etoposide, fludarabine, irinotecan, paclitaxel, rapamycin, rituximab, vincristine and the like, for example with a polytherapy such as CHOP (cyclophosphamide+doxorubicin+vincristine+prednisone), RCVP (rituximab+cyclophosphamide+vincristine+prednisone), R-CHOP (rituximab+CHOP) or DA-EPOCH-R (dose-adjusted etoposide, prednisone, vincristine, cyclophosphamide, doxorubicin and rituximab).

A composition of the invention, for example such a composition comprising ABT-263, can be administered in combination therapy with one or more therapeutic agents that include, but are not limited to, alkylating agents, angiogenesis inhibitors, antibodies, antimetabolites, antimitotics, antiproliferatives, antivirals, aurora kinase inhibitors, other apoptosis promoters (for example, Bcl-xL, Bcl-w and Bfl-1 inhibitors), activators of a death receptor pathway, Bcr-Abl kinase inhibitors, BiTE (bi-specific T-cell engager) antibodies, antibody-drug conjugates, biological response modifiers, cyclin-dependent kinase (CDK) inhibitors, cell cycle inhibitors, cyclooxygenase-2 (COX-2) inhibitors, dual variable domain binding proteins (DVDs), human epidermal growth factor receptor 2 (ErbB2 or HER/2neu) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAPB), intercalating antibiotics, kinase inhibitors, kinesin inhibitors, JAK2 inhibitors, mammalian target of rapamycin (mTOR) inhibitors, microRNAs, mitogen-activated extracellular signal-regulated kinase (MEK) inhibitors, multivalent binding proteins, non-steroidal anti-inflammatory drugs (NSAIDs), poly-ADP (adenosine diphosphate)-ribose polymerase (PARP) inhibitors, platinum chemotherapeutics, polo-like kinase (Plk) inhibitors, phosphoinositide-3 kinase (PI3K) inhibitors, proteasome inhibitors, purine analogs, pyrimidine analogs, receptor tyrosine kinase inhibitors, retinoids, deltoids, plant alkaloids, small inhibitory ribonucleic acids (siRNAs), topoisomerase inhibitors, ubiquitin ligase inhibitors, and the like.

BiTE antibodies are bi-specific antibodies that direct T-cells to attack cancer cells by simultaneously binding the two cells. The T-cell then attacks the target cancer cell. Examples of BiTE antibodies include, but are not limited to, adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (Sutton et al. (1997) J. Immunol. 158:5783-5790).

siRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand. For example, siRNAs targeting Mcl-1 have been shown to enhance the activity of ABT-263 or ABT-737 in various tumor cell lines (Tse et al. (2008) Cancer Res. 68:3421-3428 and references therein).

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy-chain DVD polypeptides and two light-chain DVD polypeptides are referred to as DVD Ig's. Each half of a DVD Ig comprises a heavy-chain DVD polypeptide, a light-chain DVD polypeptide, and two antigen binding sites. Each binding site comprises a heavy-chain variable domain and a light-chain variable domain with a total of 6 CDRs involved in antigen binding per antigen binding site.

Alkylating agents include altretamine, AMD-473, AP-5280, apaziquone, bendamustine, brostallicin, busulfan, carboquone, carmustine (BCNU), chlorambucil, Cloretazine™ (laromustine, VNP 40101M), cyclophosphamide, dacarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, treosulfan, trofosfamide and the like.

Angiogenesis inhibitors include epidermal growth factor receptor (EGFR) inhibitors, endothelial-specific receptor tyrosine kinase (Tie-2) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include Alimta™ (pemetrexed disodium, LY231514, MTA), 5-azacitidine, Xeloda™ (capecitabine), carmofur, Leustat™ (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethenylcytidine, fludarabine, 5-fluorouracil (5-FU) alone or in combination with leucovorin, Gemzar™ (gemcitabine), hydroxyurea, Alkeran™ (melphalan), mercaptopurine, 6-mercaptopurine riboside, methotrexate, mycophenolic acid, nelarabine, nolatrexed, ocfosfate, pelitrexol, pentostatin, raltitrexed, ribavirin, S-1, triapine, trimetrexate, TS-1, tiazofurin, tegafur, vidarabine, UFT and the like.

Antivirals include ritonavir, hydroxychloroquine and the like.

Aurora kinase inhibitors include ABT-348, AZD-1152, MLN-8054, VX-680, aurora A-specific kinase inhibitors, aurora B-specific kinase inhibitors, pan-aurora kinase inhibitors and the like.

Bcl-2 family protein inhibitors other than ABT-263 or compounds of Formula I herein include AT-101 ((−)gossypol), Genasense™ Bcl-2-targeting antisense oligonucleotide (G3139 or oblimersen), IPI-194, IPI-565, ABT-737, GX-070 (obatoclax) and the like.

Bcr-Abl kinase inhibitors include dasatinib (BMS-354825), Gleevec™ (imatinib) and the like.

CDK inhibitors include AZD-5438, BMI-1040, BMS-387032, CVT-2584, flavopyridol, GPC-286199, MCS-5A, PD0332991, PHA-690509, seliciclib (CYC-202 or R-roscovitine), ZK-304709 and the like.

COX-2 inhibitors include ABT-963, Arcoxia™ (etoricoxib), Bextra™ (valdecoxib), BMS-347070, Celebrex™ (celecoxib), COX-189 (lumiracoxib), CT-3, Deramaxx™ (deracoxib), JTE-522, 4-methyl-2-(3,4-dimethylphenyl)-1-(4-sulfamoylphenyl)-1H-pyrrole, MK-663 (etoricoxib), NS-398, parecoxib, RS-57067, SC-58125, SD-8381, SVT-2016, S-2474, T-614, Vioxx™ (rofecoxib) and the like.

EGFR inhibitors include ABX-EGF, anti-EGFR immunoliposomes, EGF-vaccine, EMD-7200, Erbitux™ (cetuximab), HR3, IgA antibodies, Iressa™ (gefitinib), Tarceva™ (erlotinib or OSI-774), TP-38, EGFR fusion protein, Tykerb™ (lapatinib) and the like.

ErbB2 receptor inhibitors include CP-724714, CI-1033 (canertinib), Herceptin™ (trastuzumab), Tykerb™ (lapatinib), Omnitarg™ (2C4, petuzumab), TAK-165, GW-572016 (ionafamib), GW-282974, EKB-569, PI-166, dHER2 (HER2 vaccine), APC-8024 (HER2 vaccine), anti-HER/2neu bispecific antibody, B7.her2IgG3, AS HER2 trifunctional bispecific antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17AAG, CNF-101, CNF-1010, CNF-2024, 17-DMAG, geldanamycin, IPI-504, KOS-953, Mycograb™ (human recombinant antibody to HSP-90), nab-17AAG, NCS-683664, PU24FC1, PU-3, radicicol, SNX-2112, STA-9090, VER-49009 and the like.

Inhibitors of apoptosis proteins include HGS-1029, GDC-0145, GDC-0152, LCL-161, LBW-242 and the like.

Antibody-drug conjugates include anti-CD22-MC-MMAF, anti-CD22-MC-MMAE, anti-CD22-MCC-DM1, CR-011-vcMMAE, PSMA-ADC, MEDI-547, SGN-19A, SGN-35, SGN-75 and the like.

Activators of death receptor pathway include TRAIL and antibodies or other agents that target TRAIL or death receptors (e.g., DR4 and DR5) such as apomab, conatumumab, ETR2-ST01, GDC0145 (lexatumumab), HGS-1029, LBY-135, PRO-1762, trastuzumab and the like.

Kinesin inhibitors include Eg5 inhibitors such as AZD-4877 and ARRY-520, CENPE inhibitors such as GSK-923295A, and the like.

JAK2 inhibitors include CEP-701 (lesaurtinib), XL019, NCB-018424 and the like.

MEK inhibitors include ARRY-142886, ARRY-438162, PD-325901, PD-98059 and the like.

mTOR inhibitors include AP-23573, CCl-779, everolimus, RAD-001, rapamycin, temsirolimus, ATP-competitive TORC1/TORC2 inhibitors, including PI-103, PP242, PP30 and Torin 1, and the like.

Non-steroidal anti-inflammatory drugs include Amigesic™ (salsalate), Dolobid™ (diflunisal), Motrin™ (ibuprofen), Orudis™ (ketoprofen), Relafen™ (nabumetone), Feldene™ (piroxicam), ibuprofen cream, Aleve™ and Naprosyn™ (naproxen), Voltaren™ (diclofenac), Indocin™ (indomethacin), Clinoril™ (sulindac), Tolectin™ (tolmetin), Lodine™ (etodolac), Toradol™ (ketorolac), Daypro™ (oxaprozin) and the like.

PDGFR inhibitors include CP-673451, CP-868596 and the like.

Platinum chemotherapeutics include cisplatin, Eloxatin™ (oxaliplatin), eptaplatin, lobaplatin, nedaplatin, Paraplatin™ (carboplatin), picoplatin, satraplatin and the like.

Polo-like kinase inhibitors include BI-2536 and the like.

Phosphoinositide-3 kinase inhibitors include wortmannin, LY-294002, XL-147, CAL-120, ONC-21, AEZS-127, ETP-45658, PX-866, GDC-0941, BGT226, BEZ235, XL765 and the like.

Thrombospondin analogs include ABT-510, ABT-567, ABT-898, TSP-1 and the like.

VEGFR inhibitors include Avastin™ (bevacizumab), ABT-869, AEE-788, Angiozyme™ (a ribozyme that inhibits angiogenesis (Ribozyme Pharmaceuticals (Boulder, Colo.) and Chiron (Emeryville, Calif.)), axitinib (AG-13736), AZD-2171, CP-547632, IM-862, Macugen™ (pegaptanib), Nexavar™ (sorafenib, BAY43-9006), pazopanib (GW-786034), vatalanib (PTK-787 or ZK-222584), Sutent™ (sunitinib or SU-11248), VEGF trap, Zactima™ (vandetanib or ZD-6474) and the like.

Antibiotics include intercalating antibiotics such as aclarubicin, actinomycin D, amrubicin, annamycin, Adriamycin™ (doxorubicin), Blenoxane™ (bleomycin), daunorubicin, Caelyx™ and Myocet™ (liposomal doxorubicin), elsamitrucin, epirubicin, glarubicin, idarubicin, mitomycin C, nemorubicin, neocarzinostatin, peplomycin, pirarubicin, rebeccamycin, stimalamer, streptozocin, Valstar™ (valrubicin), zinostatin and the like.

Topoisomerase inhibitors include aclarubicin, 9-aminocamptothecin, amonafide, amsacrine, becatecarin, belotecan, BN-80915, Camptosar™ (irinotecan hydrochloride), camptothecin, Cardioxane™ (dexrazoxane), diflomotecan, edotecarin, Ellence™ and Pharmorubicin™ (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include Avastin™ (bevacizumab), CD40-specific antibodies, chTNT-1/B, denosumab, Erbitux™ (cetuximab), Humax-CD4™ (zanolimumab), IGF1R-specific antibodies, lintuzumab, Panorex™ (edrecolomab), Rencarex™ (WX G250), Rituxan™ (rituximab), ticilimumab, trastuzumab, CD20 antibodies types I and II and the like.

Hormonal therapies include Arimidex™ (anastrozole), Aromasin™ (exemestane), arzoxifene, Casodex™ (bicalutamide), Cetrotide™ (cetrorelix), degarelix, deslorelin, Desopan™ (trilostane), dexamethasone, Drogenil™ (flutamide), Evista™ (raloxifene), Afema™ (fadrozole), Fareston™ (toremifene), Faslodex™ (fulvestrant), Femara™ (letrozole), formestane, glucocorticoids, Hectorol™ (doxercalciferol), Renagel™ (sevelamer carbonate), lasofoxifene, leuprolide acetate, Megace™ (megestrol), Mifeprex™ (mifepristone), Nilandron™ (nilutamide), tamoxifen including Nolvadex™ (tamoxifen citrate), Plenaxis™ (abarelix), prednisone, Propecia™ (finasteride), rilostane, Suprefact™ (buserelin), luteinizing hormone releasing hormone (LHRH) including Trelstar™ (triptorelin), histrelin including Vantas™ (histrelin implant), Modrastane™ (trilostane), Zoladex™ (goserelin) and the like.

Deltoids and retinoids include seocalcitol (EB1089 or CB1093), lexacalcitol (KH1060), fenretinide, Panretin™ (alitretinoin), tretinoin including Atragen™ (liposomal tretinoin), Targretin™ (bexarotene), LGD-1550 and the like.

PARP inhibitors include ABT-888, olaparib, KU-59436, AZD-2281, AG-014699, BSI-201, BGP-15, INO-1001, ONO-2231 and the like.

Plant alkaloids include vincristine, vinblastine, vindesine, vinorelbine and the like.

Proteasome inhibitors include Velcade™ (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, Actimmune™ (interferon gamma-1b), interferon gamma-nl, combinations thereof and the like. Other agents include Alfaferone (IFN-α), BAM-002 (oxidized glutathione), Beromun™ (tasonermin), Bexxar™ (tositumomab), Campath™ (alemtuzumab), CTLA4 (cytotoxic lymphocyte antigen 4), dacarbazine, denileukin, epratuzumab, Granocyte™ (lenograstim), lentinan, leukocyte alpha interferon, imiquimod, MDX-010 (anti-CTLA-4), melanoma vaccine, mitumomab, molgramostim, Mylotarg™ (gemtuzumab ozogamicin), Neupogen™ (filgrastim), OncoVAC-CL, Ovarex™ (oregovomab), pemtumomab (Y-muHMFG1), Provenge™ (sipuleucel-T), sargaramostim, sizofuran, teceleukin, Theracys™ (BCG or Bacillus Calmette-Guerin), ubenimex, Virulizin™ (immunotherapeutic, Lorus Pharmaceuticals), Z-100 (Specific Substance of Maruyama or SSM), WF-10 (tetrachlorodecaoxide or TCDO), Proleukin™ (aldesleukin), Zadaxin™ (thymalfasin), Zenapax™ (daclizumab), Zevalin™ (90Y-ibritumomab tiuxetan) and the like. Biological response modifiers are agents that modify defense mechanisms of living organisms or biological responses, such as survival, growth or differentiation of tissue cells to direct them to have anti-tumor activity, and include krestin, lentinan, sizofuran, picibanil, PF-3512676 (CpG-8954), ubenimex and the like.

Pyrimidine analogs include cytarabine (cytosine arabinoside, ara C or arabinoside C), doxifluridine, Fludara™ (fludarabine), 5-FU (5-fluorouracil), floxuridine, Gemzar™ (gemcitabine), Tomudex™ (raltitrexed), triacetyluridine, Troxatyl™ (troxacitabine) and the like.

Purine analogs include Lanvis™ (thioguanine), Purinethol™ (mercaptopurine) and the like.

Antimitotic agents include batabulin, epothilone D (KOS-862), N-(2-((4-hydroxy-phenyl)amino)pyridin-3-yl)-4-methoxybenzenesulfonamide, ixabepilone (BMS-247550), paclitaxel, Taxotere™ (docetaxel), larotaxel (PNU-100940, RPR-109881 or XRP-9881), patupilone, vinflunine, ZK-EPO (synthetic epothilone) and the like.

Ubiquitin ligase inhibitors include MDM2 inhibitors such as nutlins, NEDD8 inhibitors such as MLN4924, and the like.

Compositions of this invention can also be used as radiosensitizers that enhance the efficacy of radiotherapy. Examples of radiotherapy include, but are not limited to, external beam radiotherapy (XBRT), teletherapy, brachytherapy, sealed-source radiotherapy, unsealed-source radiotherapy and the like.

Additionally or alternatively, a composition of the present invention can be administered in combination therapy with one or more antitumor or chemotherapeutic agents selected from Abraxane™ (ABI-007), ABT-100 (farnesyl transferase inhibitor), Advexin™ (Ad5CMV-p53 vaccine or contusugene ladenovec), Altocor™ or Mevacor™ (lovastatin), Ampligen™ (poly(I)-poly(C12U), a synthetic RNA), Aptosyn™ (exisulind), Aredia™ (pamidronic acid), arglabin, L-asparaginase, atamestane (1-methyl-3,17-dione-androsta-1,4-diene), Avage™ (tazarotene), AVE-8062 (combretastatin derivative), BEC2 (mitumomab), cachectin or cachexin (tumor necrosis factor), Canvaxin™ (melanoma vaccine), CeaVac™ (cancer vaccine), Celeuk™ (celmoleukin), histamine including Ceplene™ (histamine dihydrochloride), Cervarix™ (AS04 adjuvant-adsorbed human papilloma virus (HPV) vaccine), CHOP (Cytoxan™ (cyclophosphamide)+Adriamycin™ (doxorubicin)+Oncovin™ (vincristine)+prednisone), combretastatin A4P, Cypat™ (cyproterone), DAB(389)EGF (catalytic and translocation domains of diphtheria toxin fused via a His-Ala linker to human epidermal growth factor), dacarbazine, dactinomycin, Dimericine™ (T4N5 liposome lotion), 5,6-dimethylxanthenone-4-acetic acid (DMXAA), discodermolide, DX-8951f (exatecan mesylate), eniluracil (ethynyluracil), squalamine including Evizon™ (squalamine lactate), enzastaurin, EPO-906 (epothilone B), Gardasil™ (quadrivalent human papilloma virus (Types 6, 11, 16, 18) recombinant vaccine), Gastrimmune™, Genasense™ (oblimersen), GMK (ganglioside conjugate vaccine), GVAX™ (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, Junovan™ and Mepact™ (mifamurtide), lonafarnib, 5,10-methylenetetrahydro folate, miltefo sine (hexadecylphosphocholine), Neovastat™ (AE-941), Neutrexin™ (trimetrexate glucuronate), Nipent™ (pentostatin), Onconase™ (ranpirnase, a ribonuclease enzyme), Oncophage™ (vitespen, melanoma vaccine treatment), OncoVAX™ (IL-2 vaccine), Orathecin™ (rubitecan), Osidem™ (antibody-based cell drug), Ovarex™ MAb (murine monoclonal antibody), paclitaxel albumin-stabilized nanoparticle, paclitaxel, Pandimex™ (aglycone saponins from ginseng comprising 20(S)-protopanaxadiol (aPPD) and 20(S)-protopanaxatriol (aPPT)), panitumumab, Panvac™-VF (investigational cancer vaccine), pegaspargase, peginterferon alfa (PEG interferon A), phenoxodiol, procarbazine, rebimastat, Removab™ (catumaxomab), Revlimid™ (lenalidomide), RSR13 (efaproxiral), Somatuline™ LA (lanreotide), Soriatane™ (acitretin), staurosporine (Streptomyces staurospores), talabostat (PT100), Targretin™ (bexarotene), Taxoprexin™ (docosahexaenoic acid (DHA)+paclitaxel), Telcyta™ (canfosfamide, TLK-286), Temodar™ (temozolomide), tesmilifene, tetrandrine, thalidomide, Theratope™ (STn-KLH vaccine), Thymitaq™ (nolatrexed dihydrochloride), TNFerade™ (adenovector: DNA carrier containing the gene for tumor necrosis factor-α), Tracleer™ or Zavesca™ (bosentan), TransMID-107R™ (KSB-311, diphtheria toxins), tretinoin (retin-A), Trisenox™ (arsenic trioxide), Ukrain™ (derivative of alkaloids from the greater celandine plant), Virulizin™, Vitaxin™ (anti-αvβ3 antibody), Xcytrin™ (motexafin gadolinium), Xinlay™ (atrasentan), Xyotax™ (paclitaxel poliglumex), Yondelis™ (trabectedin), ZD-6126 (N-acetylcolchinol-O-phosphate), Zinecard™ (dexrazoxane), zoledronic acid, zorubicin and the like.

In one embodiment, a composition of the invention, for example such a composition comprising ABT-263, is administered in a therapeutically effective amount to a subject in need thereof to treat a disease during which is overexpressed one or more of antiapoptotic Bcl-2 protein, antiapoptotic Bcl-X_{L} protein and antiapoptotic Bcl-w protein.

In another embodiment, a composition of the invention, for example such a composition comprising ABT-263, is administered in a therapeutically effective amount to a subject in need thereof to treat a disease of abnormal cell growth and/or dysregulated apoptosis.

Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In a more particular embodiment, a composition of the invention, for example such a composition comprising a ABT-263-containing solid dispersion, is administered in a therapeutically effective amount to a subject in need thereof to treat bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer or spleen cancer.

According to any of these embodiments, the composition is administered in monotherapy or in combination therapy with one or more additional therapeutic agents.

For example, a method for treating mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof in a subject comprises administering to the subject therapeutically effective amounts of (a) a composition of the invention, for example such a composition comprising ABT-263, and (b) one or more of etoposide, vincristine, CHOP, rituximab, rapamycin, R-CHOP, RCVP, DA-EPOCH-R or bortezomib.

In particular embodiments, a composition of the invention, for example such a composition comprising ABT-263, is administered in a therapeutically effective amount to a subject in need thereof in combination therapy with etoposide, vincristine, CHOP, rituximab, rapamycin, R-CHOP, RCVP, DA-EPOCH-R or bortezomib in a therapeutically effective amount, for treatment of a lymphoid malignancy such as B-cell lymphoma or non-Hodgkin's lymphoma.

In other particular embodiments, a composition of the invention, for example such a composition comprising ABT-263, is administered in a therapeutically effective amount to a subject in need thereof in monotherapy or in combination therapy with etoposide, vincristine, CHOP, rituximab, rapamycin, R-CHOP, RCVP, DA-EPOCH-R or bortezomib in a therapeutically effective amount, for treatment of chronic lymphocytic leukemia or acute lymphocytic leukemia.

The present invention also provides a method for maintaining in bloodstream of a human cancer patient a therapeutically effective plasma concentration of ABT-263 and/or one or more metabolites thereof, comprising administering to the subject a solid dispersion of ABT-263 or a pharmaceutically acceptable salt, prodrug, salt of a prodrug or metabolite thereof (for example ABT-263 free base or ABT-263 bis-HCl) in essentially non-crystalline form in a matrix that comprises a pharmaceutically acceptable water-soluble polymeric carrier and a pharmaceutically acceptable surfactant, in a dosage amount equivalent to about 50 to about 500 mg ABT-263 per day, at an average dosage interval of about 3 hours to about 7 days.

What constitutes a therapeutically effective plasma concentration depends inter alia on the particular cancer present in the patient, the stage, severity and aggressiveness of the cancer, and the outcome sought (e.g., stabilization, reduction in tumor growth, tumor shrinkage, reduced risk of metastasis, etc.). It is strongly preferred that, while the plasma concentration is sufficient to provide benefit in terms of treating the cancer, it should not be sufficient to provoke an adverse side-effect to an unacceptable or intolerable degree.

For treatment of cancer in general and of a lymphoid malignancy such as non-Hodgkin's lymphoma in particular, the plasma concentration of ABT-263 should in most cases be maintained in a range of about 0.5 to about 10 μg/ml. Thus, during a course of ABT-263 therapy, the steady-state Cₘₐₓ should in general not exceed about 10 μg/ml, and the steady-state Cₘᵢₙ should in general not fall below about 0.5 μg/ml. It will further be found desirable to select, within the ranges provided above, a daily dosage amount and average dosage interval effective to provide a Cₘₐₓ/Cₘᵢₙ ratio not greater than about 5, for example not greater than about 3, at steady-state. It will be understood that longer dosage intervals will tend to result in greater Cₘₐₓ/Cₘᵢₙ ratios. Illustratively, at steady-state, an ABT-263 Cₘₐₓ of about 3 to about 8 μg/ml and Cₘᵢₙ of about 1 to about 5 μg/ml can be targeted by the present method.

A daily dosage amount effective to maintain a therapeutically effective ABT-263 plasma level is, according to the present embodiment, about 50 to about 500 mg. In most cases a suitable daily dosage amount is about 200 to about 400 mg. Illustratively, the daily dosage amount can be for example about 50, about 100, about 150, about 200, about 250, about 300, about 350, about 400, about 450 or about 500 mg.

An average dosage interval effective to maintain a therapeutically effective ABT-263 plasma level is, according to the present embodiment, about 3 hours to about 7 days. In most cases a suitable average dosage interval is about 8 hours to about 3 days, or about 12 hours to about 2 days. A once-daily (q.d.) administration regimen is often suitable.

For the present embodiment, ABT-263 is illustratively present in the pharmaceutical composition in the form of ABT-263 free base or ABT-263 bis-HCl, more particularly ABT-263 free base. Any ABT-263 composition of the present invention, as defined more fully above, can be used.

As in other embodiments, administration according to the present embodiment can be with or without food, i.e., in a non-fasting or fasting condition. It is generally preferred to administer the present compositions to a non-fasting patient.

### EXAMPLES

The following examples are merely illustrative, and do not limit this disclosure in any way. Trademarked ingredients used in the examples, which can be substituted with comparable ingredients from other suppliers, include:

ProSolv™ HD90 of JRS Pharma: siliconized microcystalline cellulose

Span™ 20 of Croda International PLC: sorbitan monolaurate

Tween™ 20 of Uniqema: polysorbate 20 surfactant;

Tween™ 80 of Uniqema: polysorbate 80 surfactant.

All ABT-263 amounts, including concentrations and doses, given in the examples are expressed as free base equivalent doses unless expressly stated otherwise. Where ABT-263 is administered as bis-HCl salt, 1.076 mg ABT-263 bis-HCl provides 1 mg ABT-263 free base equivalent.

### Example 1

### Preparation of Solid Dispersions of ABT-263 bis-HCl

ABT-263 bis-HCl crystalline salt was mixed with a surfactant and a water-soluble polymer in the following weight ratios:

10.8% ABT-263 salt (10% free base equivalent); 10% surfactant; 79.2% polymer

21.5% ABT-263 salt (20% free base equivalent); 10% surfactant; 68.5% polymer

32.3% ABT-263 salt (30% free base equivalent); 10% surfactant; 57.7% polymer

43% ABT-263 salt (40% free base equivalent); 10% surfactant; 47% polymer

The surfactant in different series was TPGS, Span™ 20 or Tween™ 20. The polymer in different series was copovidone (Kollidon™ VA 64), povidone K-30 or HPMC-AS.

The mixture of ingredients in each case was dissolved in methanol. The methanol was removed at 65° C. in vacuo using a Genevac™ system, and the resulting solid dispersion was allowed to cool to ambient temperature.

The solid dispersion in each case was sieved through a 40-mesh screen to provide a powder of reduced particle size. The resulting powders were used for determination of T_{g} by differential scanning calorimetry (DSC), residual solvent and moisture determination by thermogravimetric analysis (TGA), characterization of crystallinity or lack thereof by powder X-ray diffraction (PXRD), and determination of physical stability when stored at 25° C./60% relative humidity (RH) and at 40° C./75% RH.

The solid dispersion powder in each case was blended with ProSolv™ HD90, croscarmellose sodium and sodium stearyl fumarate at a weight ratio of 82:15:2:1. The resulting blend was filled into hard gelatin capsules of a size, depending on drug loading, to provide a 50 mg unit dose of ABT-263. The capsules were tested for dissolution in a pH 6.5 buffer medium containing 7.6 mM Tween™ 80, using USP apparatus II (see Example 3 below).

All tested solid dispersions of ABT-263 bis-HCl prepared as above were found to have a T_{g} in the range of 70-110° C. TGA showed that the copovidone/HPMC-AS dispersions had the lowest moisture content (2-4%) and the povidone dispersions, regardless of surfactant used, had the highest moisture content (8-10%). PXRD showed no crystallinity, i.e., the ABT-263 bis-HCl was amorphous in all solid dispersions. Only the ABT-263 bis-HCl solid dispersions prepared with HPMC-AS as the polymeric carrier showed acceptable storage stability for one month. Where povidone or copovidone was used, a tendency for deliquescence was observed in open-dish storage stability testing at both at 25° C./60% RH and at 40° C./75% RH.

### Example 2

### Preparation of Solid Dispersions of ABT-263 Free Base

ABT-263 bis-HCl crystalline salt was dissolved in acetone, and NaOH was added to convert the ABT-263 bis-HCl to free base. The NaCl by-product precipitated and was removed by filtration.

To the resulting ABT-263 free base solution in acetone were added a surfactant and a water-soluble polymer in the following weight ratios:

10% ABT-263 free base; 10% surfactant; 80% polymer

20% ABT-263 free base; 10% surfactant; 70% polymer

30% ABT-263 free base; 10% surfactant; 60% polymer

40% ABT-263 free base; 10% surfactant; 50% polymer

The surfactant in different series was TPGS, Span™ 20 or Tween™ 20. The polymer in different series was copovidone (Kollidon™ VA 64) or HPMC-AS.

The acetone was removed at 65° C. in vacuo using a Genevac™ system, and the resulting solid dispersion was allowed to cool to ambient temperature.

The solid dispersion in each case was sieved through a 40-mesh screen to provide a powder of reduced particle size. The resulting powders, as in Example 1, were used for determination of T_{g} by DSC, residual solvent and moisture determination by TGA, characterization of crystallinity or lack thereof by PXRD, and determination of physical stability when stored at 25° C./60% RH and at 40° C./75% RH.

The solid dispersion powder in each case was blended with ProSolv™, croscarmellose sodium and sodium stearyl fumarate at a weight ratio of 82:15:2:1. The resulting blend was filled into hard gelatin capsules of a size, depending on drug loading, to provide a 50 mg unit dose of ABT-263. The capsules were tested for dissolution in a pH 6.5 buffer medium containing 7.6 mM Tween™ 80 (see Example 3 below).

All tested solid dispersions of ABT-263 free base prepared as above were found to have a T_{g} in the range of 70-110° C. TGA showed that the copovidone and HPMC-AS dispersions had low moisture content (2-4%). PXRD showed no crystallinity, i.e., the ABT-263 free base was amorphous in all solid dispersions. The ABT-263 free base solid dispersions prepared with copovidone or HPMC-AS as the polymeric carrier showed acceptable storage stability for one month without any sign of deliquescence.

### Example 3

### Dissolution Profiles of Solid Dispersions

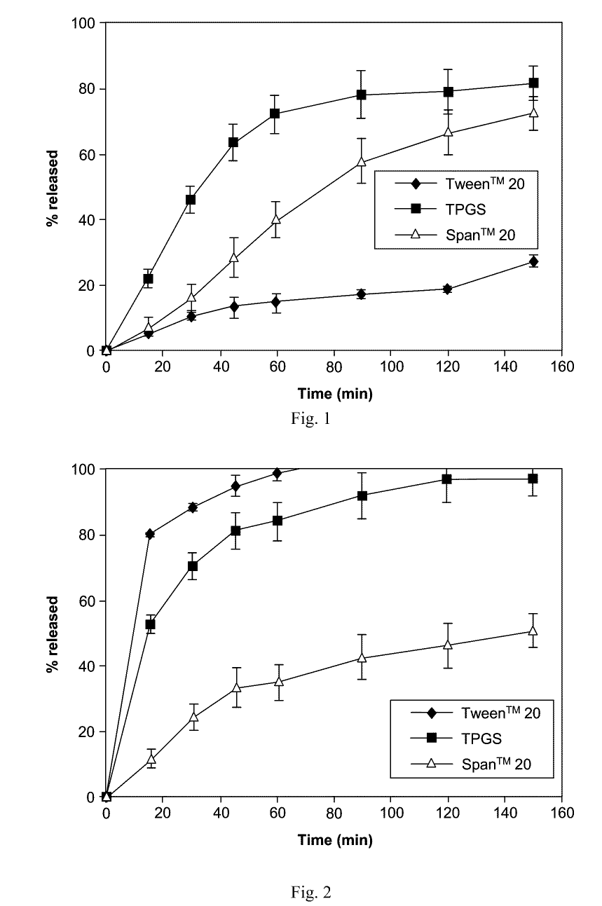

Representative dissolution (drug release) profiles in a pH 6.5 buffered medium containing 7.6 mM Tween™ 80 are shown in FIG. 1 (ABT-263 bis-HCl) and FIG. 2 (ABT-263 free base).

As shown in FIG. 1, at a 20% drug-loading level, the ABT-263 bis-HCl solid dispersions with 68.5% copovidone and 10% TPGS showed a moderate rate of drug release that plateaued at about 80% release. Release from similar dispersions having Span™ 20 or, especially, Tween™ 20 as the surfactant was much slower.

By contrast, as shown in FIG. 2, at the same 20% drug-loading level, the ABT-263 free base solid dispersions with 70% copovidone and 10% of either Tween™ 20 or TPGS showed rapid dug release. Only the Span™ 20 surfactant resulted in much slower release in the case of the free base dispersion.

Release rate was drug-loading-dependent in both ABT-263 bis-HCl and free base dispersion formulations, the 20% dispersions showing faster release than the 30% or 40% dispersions in both cases.

Unlike the analogous solid dispersion prepared from the ABT-263 free base, the solid dispersion containing ABT-263 bis-HCl, copovidone and Tween™ 20 showed shell formation. This shell formation is believed to be caused by precipitation of the drug on the surface of the capsule fill plug.

In a separate study, solid dispersions of ABT-263 bis-HCl in a copovidone matrix with and without replacement of 5% copovidone with HPMC-AS showed slower drug release in presence of HPMC-AS.

### Example 4

### Effect of Polymeric Carrier on Dissolution Profile of ABT-263 Bis-HCl Dispersions

Solid dispersions with different polymeric carriers were tested to observe impact of the polymeric carriers on dissolution rates. Four solid dispersions were prepared with ABT-263 bis-HCl salt (20% free base equivalent), 10% TPGS and the following polymeric carriers:

povidone only

50% povidone+50% copovidone

25% povidone+75% copovidone

copovidone only

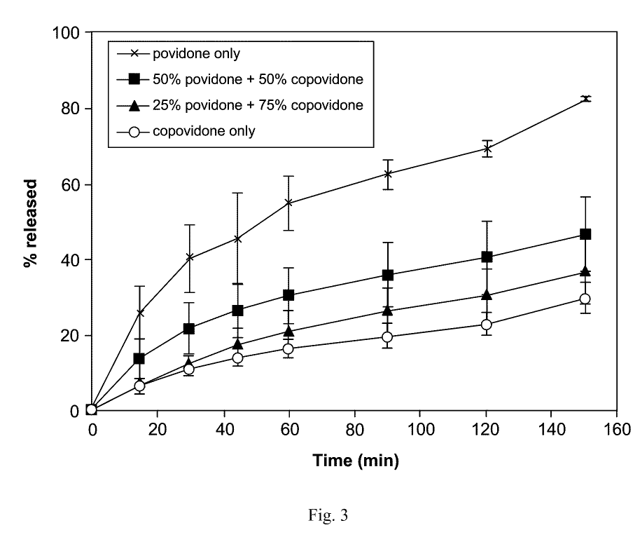

Dissolution profiles of the four solid dispersions are shown in FIG. 3. Drug release rate increased with increasing levels of povidone.

### Example 5

### Pharmacokinetics of ABT-263 bis-HCl Dispersions in a Dog Model

Single-dose pharmacokinetics of two ABT-263 solid dispersions were evaluated in non-fasted beagle dogs (n=6) after a 50 mg/kg oral dose followed by 10 ml water. Serial heparinized blood samples were obtained from a jugular vein of each animal prior to dosing and 0.25, 0.5, 1, 1.5, 2, 3, 4, 6, 9, 12, 15 and 24 hours after administration. Plasma was separated by centrifugation (2,000 rpm for 10 minutes at approximately 4° C.) and ABT-263 was isolated using protein precipitation with acetonitrile.

Two ABT-263 bis-HCl solid dispersions (those of Example 4 containing povidone only or copovidone only) were compared. The powdered dispersions were blended with ProSolv™ HD90, croscarmellose sodium and sodium stearyl fumarate in an 82:15:2:1 weight ratio and the blend filled into capsules.

ABT-263 and an internal standard were separated from each other and from co-extracted contaminants on a 50×3 mm Keystone Betasil CN™ 5 μm column with an acetonitrile/0.1% trifluoroacetic acid mobile phase (50:50 by volume) at a flow rate of 0.7 ml/min. Analysis was performed on a Sciex API3000™ biomolecular mass analyzer with a heated nebulizer interface. ABT-263 and internal standard peak areas were determined using Sciex MacQuan™ software. The plasma drug concentration of each sample was calculated by least squares linear regression analysis (non-weighted) of the peak area ratio (parent/internal standard) of the spiked plasma standards versus concentration. The plasma concentration data were submitted to multi-exponential curve fitting using WinNonlin 3 (Pharsight).

The area under the plasma concentration-time curve from 0 to t hours (time of the last measured plasma concentration) after dosing (AUC₀₋ₜ) was calculated using the linear trapezoidal rule for the plasma concentration-time profiles. The residual area extrapolated to infinity, determined as the final measured plasma concentration (Cₜ) divided by the terminal elimination rate constant (β), was added to AUC₀₋ₜ to produce the total area under the curve (AUC_{0-∞}). The bioavailability was calculated as the dose-normalized AUC_{0-∞} from oral dosing divided by the corresponding value derived from i.v. (intravenous) dosing, administered as a slow bolus to a jugular vein under light ether anesthetic.

PK parameters for the povidone-only and copovidone-only dispersions are presented in Table 1.

TABLE 1
PK parameters of solid dispersion compositions in dog (n = 6)

| | | Cₘₐₓ/D | | | AUC/D | |
|---|---|---|---|---|---|---|
| | Cₘₐₓ | μg/ml per | Tₘₐₓ | AUC | μg · h/ml | F |
| Composition | μg/ml | mg/kg | h | μg · h/ml | per mg/kg | % |
| povidone | 5.6 | 1.16 | 9.8 | 39.3 | 7.9 | 16.4 |
| copovidone | 9.6 | 1.78 | 4.5 | 64.9 | 11.9 | 24.7 |

Although the ABT-263 bis-HCl dispersion prepared with povidone was shown in Example 4 to provide a better release rate than copovidone, it had poorer bioavailability in this dog study than a comparable dispersion prepared with copovidone.

### Example 6

### Pharmacokinetics of Illustrative Solid Dispersions in a Dog Model

Single-dose pharmacokinetics of two ABT-263 solid dispersions were evaluated in non-fasted beagle dogs (n=6), following the same protocol as that of Example 5. Two ABT-263 solid dispersions (Dispersions I and II) were prepared. Dispersion I, prepared substantially according to the process of Example 2, contained 10% ABT-263 free base, 10%

TPGS and 80% copovidone. The powdered dispersion was filled into capsules without any additional ingredients to prepare Composition I. Dispersion II, prepared substantially according to the process of Example 1, contained 13.11% ABT-263 bis-HCl (12.18% free base equivalent), 15% TPGS and 71.89% povidone. The powdered dispersion was blended with ProSolv™ HD90, sodium starch glycolate and sodium stearyl fumarate in an 82:15:2:1 weight ratio and the blend filled into capsules to prepare Composition II.

PK parameters for Compositions I and II are presented in Table 2.

TABLE 2
PK parameters of solid dispersion compositions in dog (n = 6)

| | | Cₘₐₓ/D | | | AUC/D | |
|---|---|---|---|---|---|---|
| | Cₘₐₓ | μg/ml per | Tₘₐₓ | AUC | μg · h/ml | F |
| Composition | μg/ml | mg/kg | h | μg · h/ml | per mg/kg | % |
| I | 7.5 | 1.50 | 8.5 | 59.0 | 11.2 | 24.6 |
| II | 6.4 | 1.24 | 7.8 | 39.2 | 7.4 | 16.3 |

The ABT-263 bis-HCl dispersion (Composition II) prepared with povidone had poorer bioavailability in this dog study than the ABT-263 free base dispersion (Composition I) prepared with copovidone.

What is claimed is:

1. A solid dispersion comprising, in essentially non-crystalline form, a compound of Formula I 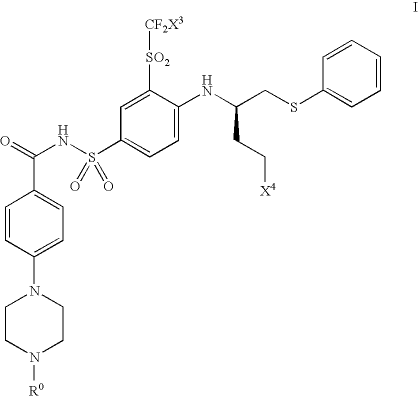
where
X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is 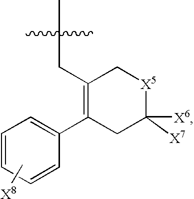
where
X⁵ is —CH₂—, —C(CH₃)₂— or —CH₂CH₂;
X⁶ and X⁷ are both —H or both methyl; and
X⁸ is fluoro, chloro, bromo or iodo;
Or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is 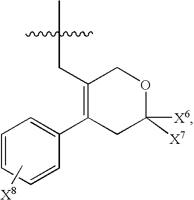
where X⁶, X⁷ and X⁸ are as above; or
(3) X⁴ is morpholin-4-yl or —N(CH₃)₂; and R⁰ is 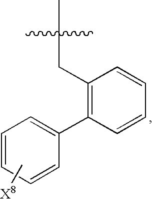
where X⁸ is as above;
or a pharmaceutically acceptable salt, prodrug, salt of a prodrug or metabolite thereof; dispersed in a solid matrix that comprises (a) at least one pharmaceutically acceptable water-soluble polymeric carrier and (b) at least one pharmaceutically acceptable surfactant.

2. The solid dispersion of claim 1, wherein the compound of Formula I is ABT-263 or a pharmaceutically acceptable salt, prodrug, salt of a prodrug or metabolite thereof.

3. The solid dispersion of claim 1, wherein the compound of Formula I is ABT-263 free base or ABT-263 bis-HCl.

4. The solid dispersion of claim 2, wherein the compound is present in an ABT-263 free base equivalent amount of about 5% to about 40% by weight.

5. The solid dispersion of claim 4, wherein the at least one polymeric carrier is present in an amount of about 40% to about 85% by weight and the at least one surfactant is present in an amount of about 5% to about 20% by weight.

6. The solid dispersion of claim 1, wherein the at least one polymeric carrier is selected from the group consisting of homopolymers and copolymers of N-vinyl lactams, cellulose esters, cellulose ethers, high molecular weight polyalkylene oxides, polyacrylates, polymethacrylates, polyacrylamides, vinyl acetate polymers, oligo- and polysaccharides and mixtures thereof.

7. The solid dispersion of claim 1, wherein the at least one polymeric carrier is selected from the group consisting of copovidone, povidone, HPMC-AS and mixtures thereof.

8. The solid dispersion of claim 1, wherein the at least one surfactant is non-ionic.

9. The solid dispersion of claim 1, wherein the at least one surfactant is selected from the group consisting of polyoxyethylene castor oil derivatives, fatty acid monoesters of sorbitan, polysorbates, poloxamers, α-tocopheryl polyethylene glycol succinate and mixtures thereof.

10. A process for preparing a solid dispersion, comprising:
(a) dissolving an active pharmaceutical ingredient (API) comprising (i) a compound of Formula I 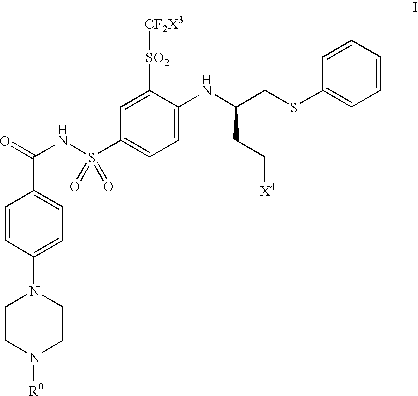
where
X³ is chloro or fluoro; and
(1) X⁴ is azepan-1-yl, morpholin-4-yl, 1,4-oxazepan-4-yl, pyrrolidin-1-yl, —N(CH₃)₂, —N(CH₃)(CH(CH₃)₂), 7-azabicyclo[2.2.1]heptan-7-yl or 2-oxa-5-azabicyclo[2.2.1]hept-5-yl; and R⁰ is 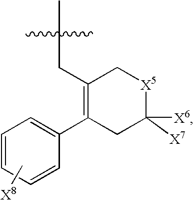
where
X⁵ is —CH₂—, —C(CH₃)₂— or —CH₂CH₂;
X⁶ and X⁷ are both —H or both methyl; and
X⁸ is fluoro, chloro, bromo or iodo;
or
(2) X⁴ is azepan-1-yl, morpholin-4-yl, pyrrolidin-1-yl, —N(CH₃)(CH(CH₃)₂) or 7-azabicyclo[2.2.1]heptan-7-yl; and R⁰ is 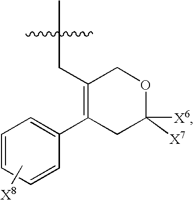
where X⁶, X⁷ and X⁸ are as above; or
(3) X⁴ is morpholin-4-yl or —N(CH₃)₂; and R⁰ is 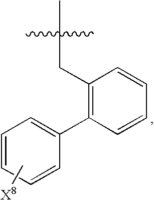
where X⁸ is as above;
or a pharmaceutically acceptable salt, prodrug, salt of a prodrug or metabolite thereof, (ii) at least one pharmaceutically acceptable water-soluble polymeric carrier and (iii) at least one pharmaceutically acceptable surfactant in a suitable solvent; and
(b) removing the solvent to provide a solid matrix comprising the at least one polymeric carrier and the at least one surfactant and having the compound or a salt, prodrug, salt of a prodrug or metabolite thereof dispersed in an essentially non-crystalline form therein.

11. The process of claim 10, wherein the compound of Formula I is ABT-263 or a pharmaceutically acceptable salt, prodrug, salt of a prodrug or metabolite thereof.

12. The process of claim 10, wherein the API comprises a compound of Formula I in a salt form; and the process further comprises converting said salt form to a free base form, prior to removing the solvent.

13. The process of claim 12, wherein said converting comprises addition of a base.

14. The process of claim 12, wherein the salt form is dissolved in the solvent and is converted therein to the free base form prior to addition of the at least one polymeric carrier and the at least one surfactant.

15. The process of claim 12, further comprising extracting a salt by-product of said conversion, prior to removing the solvent.

16. The process of claim 10, wherein the solvent is removed under heat and/or vacuum.

17. The process of claim 10, wherein the solvent comprises methanol, ethanol or acetone.

18. An orally deliverable pharmaceutical dosage form comprising the solid dispersion of claim 1.

19. A method for treating a disease characterized by apoptotic dysfunction and/or overexpression of an anti-apoptotic Bcl-2 family protein, comprising orally administering to a subject having the disease a therapeutically effective amount of the solid dispersion of claim 1.

20. The method of claim 19, wherein the disease is a neoplastic disease.

21. The method of claim 20, wherein the neoplastic disease is selected from the group consisting of cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma and combinations thereof.

22. The method of claim 20, wherein the neoplastic disease is a lymphoid malignancy.

23. The method of claim 22, wherein the lymphoid malignancy is non-Hodgkin's lymphoma.

24. The method of claim 20, wherein the neoplastic disease is chronic lymphocytic leukemia or acute lymphocytic leukemia.

25. The method of claim 19, wherein the compound of Formula I in the solid dispersion administered is ABT-263 or a pharmaceutically acceptable salt, prodrug, salt of a prodrug or metabolite thereof.

26. The method of claim 24, wherein the solid dispersion is administered in a dose of about 50 to about 500 mg ABT-263 free base equivalent per day at an average treatment interval of about 3 hours to about 7 days.

27. The method of claim 24, wherein the composition is administered once daily in a dose of about 200 to about 400 mg ABT-263 free base equivalent per day.
